(19) United States
(12) Patent Application Publication
(10) Pub. No.: US 2023/0285251 A1
(43) Pub. Date: Sep. 14, 2023

(54) Universal Hair Magic is a two-step hair and skin restoration program, comprised of an organic, proprietary formulated medicated hard soap bar and medicated serum, for all mankind.

(71) Applicant: Christine Denise McLean, Tukwila, WA (US)

(72) Inventor: Christine Denise McLean, Tukwila, WA (US)

(21) Appl. No.: 17/689,978

(22) Filed: Mar. 8, 2022

(51) Int. Cl. A61K 8/02; A61K 8/67; A61K 8/36; A61K 8/19; A61K 8/24; A61K 8/27; A61K 8/64; A61Q 5/02; A61Q 19/10

(52) U.S. Cl. CPC A61K 8/0216; A61K 8/678; A61K 8/676; A61K 8/673; A61K 8/671; A61K 8/361; A61K 8/19; A61K 8/24; A61K 8/27; A61K 8/64; A61Q 5/02; A61Q 19/10; A61K 2800/884

(57) ABSTRACT

Universal Hair Magic is the hair and skin restoration program, which meets the needs of all mankind, in two easy steps. The program contains a naturally medicated hard soap bar and medicated serum. Both the soap bar and serum are formulated with a proprietary blend of organic, food grade Cocoa Butter, Sunflower Seed and Walnut Oils. No other ingredients are included. The program is naturally rich in Vitamins A, B, C, D, E, and F; Amino Acids, Calcium, Magnesium, Phosphorus, Potassium, Protein and Zinc; Omega-3, Omega-6, Omega-9, Palmitic and Stearic Fatty acids. It's naturally Anti-aging, Anti-bacterial, Anti-cancer, Anti-dandruff, Anti-fungal, Anti-inflammatory, Anti-microbial and Antioxidant. It prevents and restores hair loss. It's the multi-tasking Hydrator, Healing, Regenerating miracle made for all ages, genders; hair and skin types; and those with or without melanin in their skin.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The two-step hair and skin restoration program Universal Hair Magic has been created for the Pharmaceutical Industry. This proprietary blended program is comprised of two components.

The first component is a natural medicated soap bar, which is for use as soap for body, eye orbit, face, feet, hands, shampoo and conditioner. The second component is a natural medicated serum, which is for treatment of cuticles, eyebrows, eye lashes, eye orbit, face, feet, hair, hands, lips, scalp and skin.

Both the soap bar and serum contain the same powerful ingredients, so healing and restoration can be experienced from head to toe. The program is formulated to be effective and safe for all age groups and genders. It's effective whether a person has melanin or no melanin in their skin; and effective for all hair and skin types.

The development of this program is solely to reduce or remove the need to purchase multiple self-care products, which are simply necessary, for each person in the household. Providing all of mankind with a premium quality hair and skin care product, that will provide immediate and permanent results.

### BRIEF SUMMARY OF THE INVENTION

Universal Hair Magic is the two-step program which has a solution for the hair and skin concerns of all mankind. This program includes a medicated hard soap bar and medicated serum. The proprietary blended formula is created using organic, food grade ingredients and supplies. The program is enriched with vitamins, fatty and amino acids, essential for optimal hair and skin health.

Fifteen months of Research and Development was conducted by the inventor and the inventor's family, exclusively using Universal Hair Magic for all hair and skin needs.

In two easy steps, Universal Hair Magic provides immediate and permanent results for preventing and restoring hair loss; contains natural UVA+UVB sun ray protection properties; and provides natural Anti-aging, Anti-bacterial, Anti-cancer, Anti-dandruff, Anti-fungal, Anti-inflammatory, Anti-microbial and Antioxidant benefits.

### BRIEF DESCRIPTION OF THE SEVERAL VIEWS OF THE DRAWING

FIG. 1A-1, which is a view of the first image, displays a black and white, frontal view photograph of the UHM (or Universal Hair Magic) medicated soap bar.

FIG. 1B-2, which is a view of the second image, displays a black and white, side view photograph of the UHM (or Universal Hair Magic) medicated soap bar.

FIG. 2-3, which is a view of the third image, displays a black and white photograph of the medicated Universal Serum, in a 1 ounce dropper bottle.

### DETAILED DESCRIPTION OF THE INVENTION

Universal Hair Magic is a two-step hair and skin restoration program, formulated to address the hair and skin care needs of all mankind, safely and effectively. This program includes a medicated hard soap bar and medicated universal serum.

The medicated soap bar is created by using the chemical cold process known as the Saponification of Fatty Acids or ‘the reaction of Fatty Acids with a base’.

The soap bar is formulated with a proprietary blend of Saponified Cocoa Butter, Sunflower Seed and Walnut Oils. The aqueous alkali or base Sodium Hydroxide is dissolved in Goat Milk before blending with Oils. There are no other ingredients contained in the soap bar. All ingredients are organic and food grade.

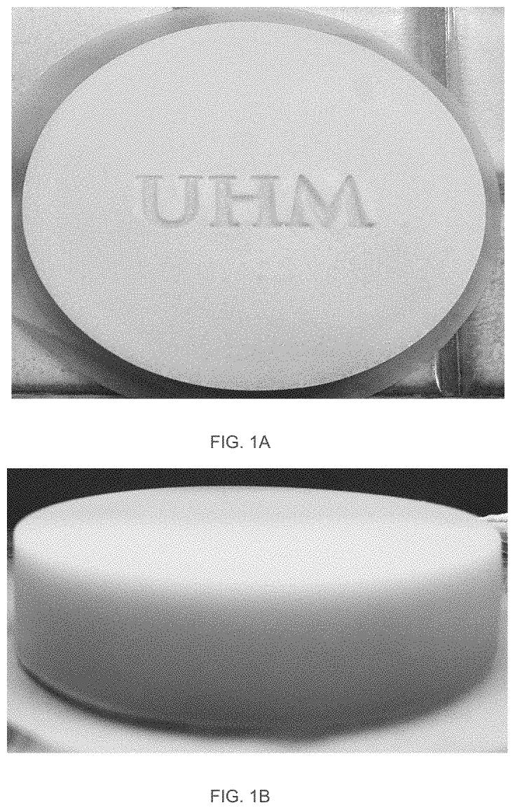

Once all ingredients are blended, round, food grade silicone molds are used to form the soap bar. Curing time for soap is three days. See FIG. 1A-1 in the Drawings section, for a photographed frontal view of soap bar; and

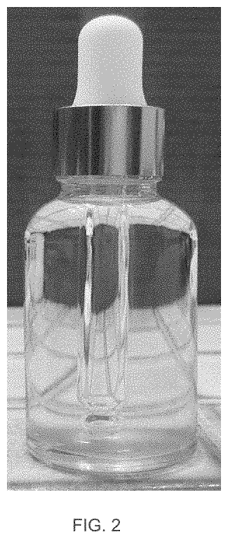

FIG. 1B-2 for a photographed side view of soap bar.

Fifteen months of Research and Development was completed by the inventor and the inventor's family. The inventor found that curing soap for three days versus 8 weeks or six months, did not produce a harder soap; or make soap bar more effective. Only three days of curing is needed.

Universal Hair Magic soap bar produces a luxurious, rich, creamy lather which gently and thoroughly cleanses the body from head to toe; penetrating the skin, to heal from within. This soap bar is for use as soap for the body, eye orbit, face, feet, hands, shampoo and conditioner.

The medicated serum is a proprietary blend of organic, food grade Cocoa Butter, Sunflower Seed and Walnut Oils. No other ingredients are included. See FIG. 2-3 in the Drawings section, for a photograph of serum in 1 oz dropper.

Universal Serum is for treatment of cuticles, eyebrows, eye lashes, eye orbit, face, feet, hair, hands, lips, scalp and skin.

The two-step Universal Hair Magic program is for all ages and genders; hair and skin types; and those with or without melanin in their skin.

1. The restoration program provides permanent healing, restoration, and or maintenance to the cellular structure of humanity, on the molecular level.

2. The program heals and restores scalp damage from the periosteum (or membrane) which covers the outer surface of the skull, through the connective tissue and skin layers; Primary and secondary skin lesions such as epidermoid cysts, shingles, and Staphylococcus aureus (MRSA) infection; and Ocular conditions such as Keratoconus (or bulging cornea); Trichiasis (or eyelid inflammation and scarring caused by eyelashes growing against the ocular surface); and Blepharitis (or inflammation of the eyelids).

3. The program heals and restores third- and fourth-degree burns caused by fire, chemicals, hot liquid, electricity or ultraviolet rays; Fungal infections such as onychomycosis (or nail fungus), ringworm, and candida (or yeastlike parasitic fungus); and aids in reducing discomfort for severe Sciatica, and intractable pain conditions such as chronic regional pain syndrome and neuropathy.

4. Vitamin A (or retinol) increases rate of healing; assists skin glands in producing sebum and supports the skin's immune system; increases production of the collagen protein; increases the rate of hair growth, encourages growth of thicker hair, while preventing loss of other hair follicles; prevents sun damage, by interrupting the process that breaks down collagen; and is needed for the growth of all cells, making this component an integral part of the program's molecular structure; ensuring humankind will obtain the results as stated in claim 2 or 3.

5. Vitamin B supplies fatty acids to the skin, improving skin tone and balancing natural oils; produces red blood cells, giving hair follicles the ability to generate new hair; stimulates keratin production in hair, increasing rate of follicle growth; provides inflammation relief, and prevents cell damage and water loss, making this component an integral part of the program's molecular structure; ensuring humankind will obtain the results as stated in claim 2 or 3.

6. Vitamin C (or ascorbic acid) stimulates collagen production; improves quality and texture of skin; helps wounds heal faster; defends against sun damage and hyperpigmentation; treats sunburn; reverses free radical damage; fades dark spots; maintains nail integrity by strengthening the skin, connective tissue, bones and blood vessel walls; heals damaged skin; and prevents dry skin, making this component an integral part of the program's molecular structure; ensuring humankind will obtain the results as stated in claim 2 or 3.

7. Vitamin D is an inhibitor of cancer cell growth; enhances the skin's immune system, by contributing to skin cell growth, repair and metabolism; helps destroy free radicals; defends against bacteria, inflammation and microbes; and is necessary for the proper function of hair and skin, making this component an integral part of the program's molecular structure; ensuring humankind will obtain the results as stated in claim 2 or 3.

8. Vitamin E is a powerful antioxidant that improves and boosts blood circulation in the body and scalp; assists in generating new skin cells; nourishes and protects skin from free radical damage; reduces photodamage; tocotrenol (part of vitamin E) fights against cancer cells; and is essential for healthy skin, making this component an integral part of the program's molecular structure; ensuring humankind will obtain the results as stated in claim 2 or 3.

9. Vitamin F protects and fortifies the skin barrier, helping to defend against ultraviolet rays and air pollutants; defends against inflammation; regulates healthy cell function and excessive water loss; helps maintain the health of cell membranes; improves nutrient use; also establishes and controls cellular metabolism, making this component an integral part of the program's molecular structure; ensuring humankind will obtain the results as stated in claim 2 or 3.

10. Calcium is an essential mineral necessary to help create the structural components of hair, skin and nails; regulates sebum production, providing hydration for skin; is a necessary part of wound healing and skin-cell regeneration, due to its important regulatory role of cell turnover within the epidermis; and helps improve skin barrier function, making this component an integral part of the program's molecular structure; ensuring humankind will obtain the results as stated in claim 2 or 3.

11. Magnesium increases blood flow in scalp, preventing hair loss and triggering hair growth; is involved in protein synthesis, helping to create strong nail plates; is an enzyme and mineral regulator; regulates cellular regeneration and repair, protecting skin from external damage; regulates calcium movement and buildup, by dissolving calcium deposits transferred to hair follicles and skin; fights inflammation and dandruff, making this component an integral part of the program's molecular structure; ensuring humankind will obtain the results as stated in claim 2 or 3.

12. Phosphorus is responsible for promoting the growth, maintenance and repair of all tissues and cells; filters out toxins, making room for new, healthy cells in every part of the body —including hair, skin and nails, making this component an integral part of the program's molecular structure; ensuring humankind will obtain the results as stated in claim 2 or 3.

13. Potassium helps deliver vital nutrients to the hair and scalp; provides hydration for skin cells; protects against ultraviolet rays; supports and promotes rapid cell renewal, making this component an integral part of the program's molecular structure; ensuring humankind will obtain the results as stated in claim 2 or 3.

14. Protein and amino acids are essential for healing and repairing damaged tissue, including that of hair, skin and nails; protein supplies the body with the amino acids it requires to make keratin (one of the structural proteins in skin, hair and nails); also increases cell turnover, making this component an integral part of the program's molecular structure; ensuring humankind will obtain the results as stated in claim 2 or 3.

15. Zinc has a role in the production of collagen, accelerating the healing process of wounds and burns; helps prevent hair loss; is an essential mineral necessary for cellular metabolism, immune function and wound healing; also provides healing for skin lesions, making this component an integral part of the program's molecular structure; ensuring humankind will obtain the results as stated in claim 2 or 3.

16. Omega-3 Fatty Acid (or Alpha-Linoleic Acid) prevents scalp damage and infections; increases hair growth, preventing breakage and loss; provides hydration to the skin; protects skin cells against sun-induced inflammation or damage from ultraviolet A and ultraviolet B rays, making this component an integral part of the program's molecular structure; ensuring humankind will obtain the results as stated in claim 2 or 3.

17. Omega-6 Fatty Acid (or Linoleic Acid) is vital to human health, as it helps to maintain bone health and regulate metabolism; stimulates hair and skin growth; helps decrease soreness in the skin; helps produce the skin's natural oil barrier; and enhances the development of new skin cells, making this component an integral part of the program's molecular structure; ensuring humankind will obtain the results as stated in claim 2 or 3.

18. Omega-9 Fatty Acid (or Oleic Acid) helps to control water loss in skin; also fights against inflammation and oxidation stress, making this component an integral part of the program's molecular structure; ensuring humankind will obtain the results as stated in claim 2 or 3.
